(19) United States
(12) Patent Application Publication
(10) Pub. No.: US 2011/0136082 A1
(43) Pub. Date: Jun. 9, 2011

(54) ELECTRONIC AID

(76) Inventor: Robene Dutta, London (GB)

(21) Appl. No.: 12/989,426

(22) PCT Filed: Apr. 24, 2009
(86) PCT No.: PCT/GB2009/001040

§ 371 Date: Feb. 22, 2011

(30) Foreign Application Priority Data
Apr. 25, 2008 (GB) 0807488.2
Nov. 25, 2008 (GB) 0821493.4

(51) Int. Cl. A61F 9/08; G09B 21/00

(52) U.S. Cl. 434/114; 434/116

(57) ABSTRACT

An electronic aid which comprises a device (1) which is configured to record and play back sound clips. The device (1) incorporates a reader (10) which is configured to read identifiers. The device (1) can be used in combination with a data carrier which may be provided with a plurality of different identifiers. A user can record sound clips and associate each sound clip with a corresponding identifier. The device (1) is configured to play back a sound clip when the reader (10) reads the corresponding identifier.

### DESCRIPTION OF THE INVENTION

THE PRESENT INVENTION relates to an electronic aid, and more particularly relates to an electronic aid for assisting a visually impaired or blind person.

A visually impaired person can face significant difficulty in carrying out certain day to day activities which are otherwise carried out easily by an able-sighted person. For instance, it can be difficult for a visually impaired person to distinguish between objects such as food tins which have the same shape and texture. A visually impaired person, for example, might not be able to distinguish between tinned food for human consumption and tinned pet food if the tins are of the same shape and texture.

Another activity which can be difficult for a visually impaired person is planning and time organisation. An able-sighted person can use a diary to plan activities whereas a visually impaired person usually cannot because it can be difficult for a visually impaired person to read and write diary entries in the correct days.

Accordingly, there is a need for a system which can assist a visually impaired person in carrying out day to day activities.

The present invention seeks to provide an improved electronic aid.

According to one aspect of the present invention, there is provided an electronic aid comprising a device which incorporates: a microphone for receiving an audio signal; an identifier reader for reading an identifier; a processing arrangement for receiving the audio signal from the microphone and creating a storable sound clip therefrom and for generating link data which defines a link between the sound clip and an identifier; a memory for storing the sound clip and link data corresponding to the sound clip; and an audio output; the device being configured to output the sound clip stored in the memory via the audio output in response to the identifier reader reading an identifier which is linked to the sound clip by corresponding link data stored in the memory.

According to another aspect of the present invention, there is provided an aid for use by a blind or visually impaired person, the aid comprising a plurality of labels which are each provided with an attachment means to allow the label to be attached to an object, wherein at least two of the labels are provided with a respective different identifier; and a device which incorporates: a microphone for receiving an audio signal; an identifier reader for reading an identifier provided on one of the labels; a processing arrangement for receiving an audio signal from the microphone and creating a storable sound clip therefrom and for generating link data which defines a link between the sound clip and an identifier read by the identifier reader; a memory for storing the sound clip and link data corresponding to the sound clip; and an audio output; the device being configured to output the sound clip stored in the memory via the audio output in response to the identifier reader reading the identifier which is linked to the sound clip by corresponding link data stored in the memory.

Preferably, the aid further comprises at least one data carrier which is provided with at least one identifier.

Conveniently, the at least one data carrier is a label which is provided with an attachment means to allow the label to be attached to an object.

In one embodiment, the attachment means is an adhesive layer.

In another embodiment, the attachment means is one of a hook layer and a loop layer of a hook and loop attachment arrangement.

Preferably, the aid comprises a plurality of data carriers and wherein at least two of the data carriers are each provided with a different identifier.

Conveniently, the at least one data carrier is a document which is provided with a plurality of different identifiers at different positions on the document.

Advantageously, the at least one data carrier is a book which is provided with a plurality of different identifiers on different parts of the book.

Preferably, each identifier is unique to its position in the book.

Conveniently, the device is configured to store in the memory a link between each of the identifiers and a part of a sound clip and to output a part of the sound clip in response to the identifier reader reading the identifier linked to the part of the sound clip.

Advantageously, the device is configured to output parts of a sound clip successively while the identifier reader reads a plurality of the identifiers successively.

Preferably, the device is configured to stop outputting parts of the sound clip if the identifier reader stops reading the identifiers successively.

Conveniently, the book is a diary and each identifier is provided on a respective part of the diary which is indicative of a date or a time.

Advantageously, the at least one data carrier is provided with printed text and/or graphics in addition to the or each identifier.

Preferably, the at least one data carrier is provided with symbols which can be detected by human touch.

Conveniently, the symbols are Braille characters.

In one embodiment, the or each identifier is a coded dot pattern.

Preferably the or each identifier is a plurality of identical coded dot patterns which are spaced apart from one another.

Preferably, the coded dot pattern is printed.

In another embodiment, the coded dot pattern is not visible to the naked eye.

In a further embodiment, the coded dot pattern is not detectable by human touch.

In a yet further embodiment, the coded dot pattern is detectable by human touch.

In one embodiment, the coded dot pattern is defined by at least one Braille character.

In another embodiment, the or each identifier is one of text and drawings which are visible to the naked eye.

In a further embodiment, the identifier is magnetically encoded information.

Preferably, the device is configured to store a plurality of sound clips and a corresponding plurality of link data.

Conveniently, the device is substantially pen-shaped.

Advantageously, the audio output incorporates a loudspeaker.

Preferably, the audio output incorporates an earphone/headphone connection.

According to a further aspect of the present invention, there is provided a method of storing information comprising receiving an audio signal with a microphone, processing the audio signal with a processing arrangement to create a storable sound clip, reading an identifier provided on a data carrier using an identifier reader, generating link data which defines a link between the sound clip and the identifier, storing the sound clip and the link data corresponding to the sound clip in a memory.

According to a yet further aspect of the present invention, there is provided a method of labelling an object comprising: attaching a label to an object, the label being provided with an identifier, reading the identifier using an identifier reader provided on a device, and recording a sound clip using the device, the device storing the sound clip and generating and storing link data which defines a link between the sound clip and the identifier so that the sound clip can be played back via an audio output in the device when the identifier reader reads the identifier when the device is subsequently activated in a playback mode.

In order that the invention may be more readily understood, and so that further features thereof may be appreciated, embodiments of the invention will now be described, by way of example, with reference to the accompanying drawings in which:

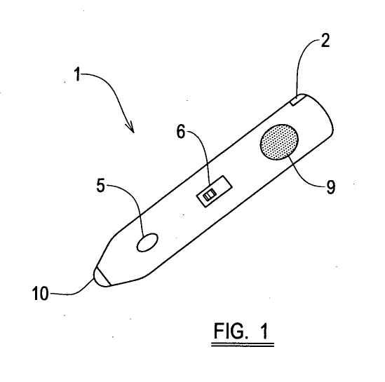

FIG. 1 is a diagrammatic view of a preferred embodiment of the invention,

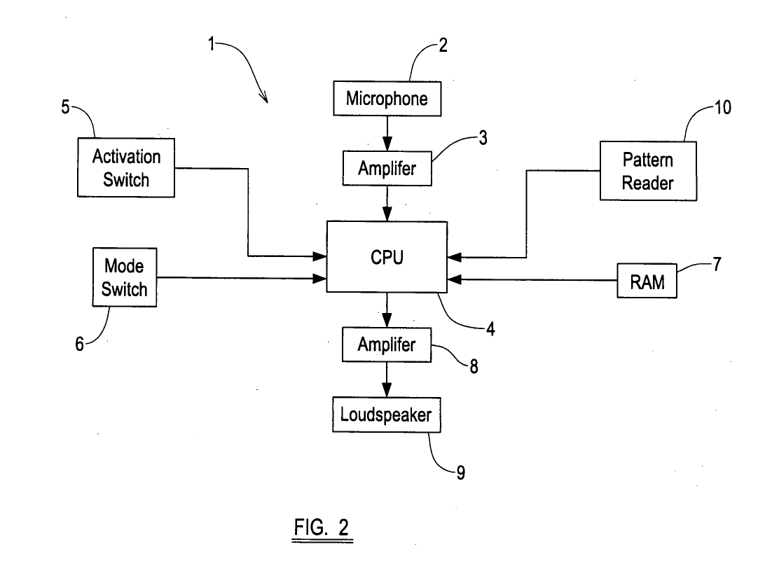

FIG. 2 is a block diagram showing components of the preferred embodiment of the invention,

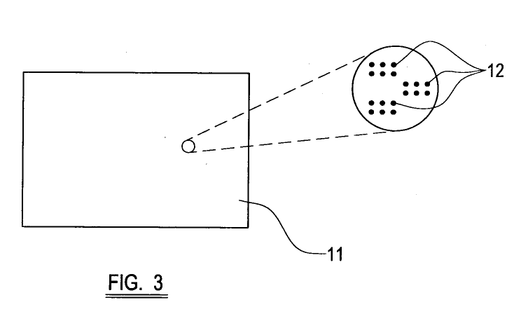

FIG. 3 is a diagrammatic view of a label for use with an embodiment of the invention with part of the label being shown magnified,

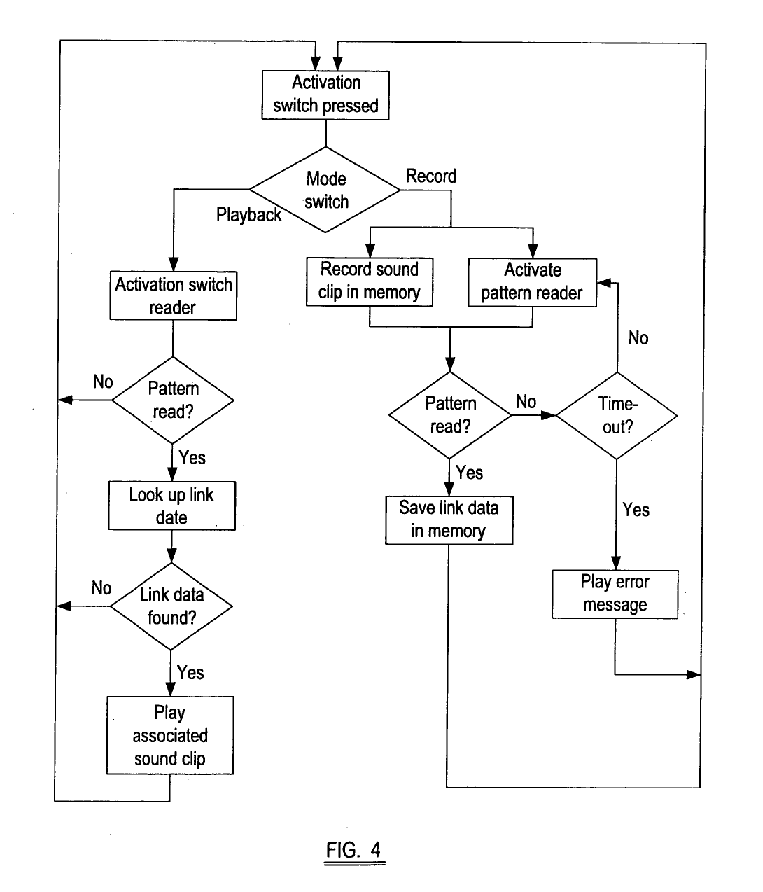

FIG. 4 is a flow diagram showing the steps of operation of the preferred embodiment of the invention,

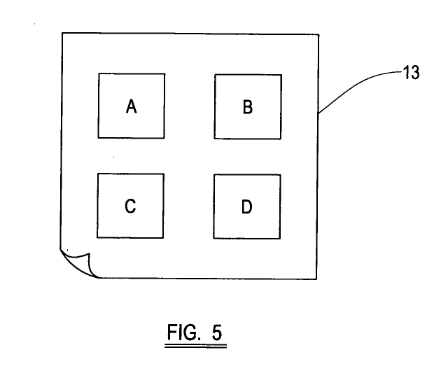

FIG. 5 is a diagrammatic view of a document for use with an embodiment of the invention,

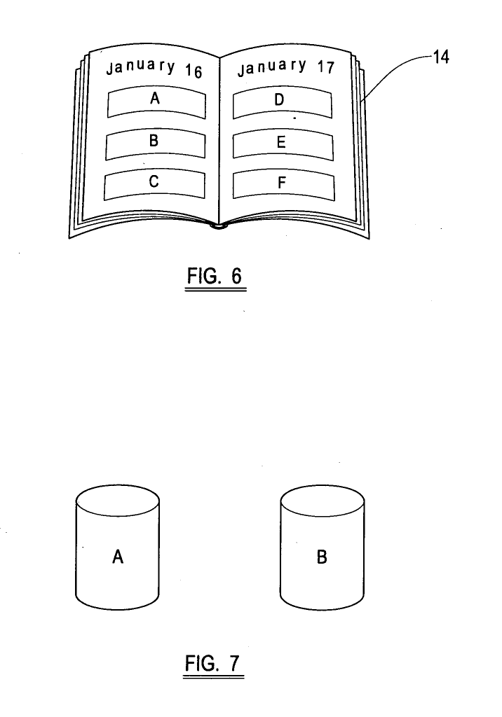

FIG. 6 is a diagrammatic view of a diary for use with an embodiment of the invention, and

FIG. 7 is a diagrammatic view of products for use with an embodiment of the invention.

Referring initially to FIGS. 1 and 2 of the accompanying drawings, an electronic aid in accordance with a preferred embodiment of the invention comprises a device 1 which is substantially pen-shaped and dimensioned to be received in the hand of a user. The device 1 incorporates a microphone 2 which is connected via a microphone amplifier 3 to a processing arrangement in the form of a central processing unit 4. The central processing unit 4 is connected via an output amplifier 8 to an audio output in the form of a loudspeaker 9.

In other embodiments, the audio output incorporates an earphone/headphone connection instead of or in addition to the loudspeaker 9 to allow a user to connect earphones or headphones to the device. In these embodiments, the device 1 can be configured to mute the loudspeaker 9 when earphones or headphones are connected to the device 1.

An activation push switch 5 and a mode selection switch 6 are connected to the central processing unit 4. The mode switch 6 can be switched by a user between a record and a playback position. The mode switch may thus be used to select the mode of operation of the device 1 from either a record mode or a playback mode. The device incorporates a power source (not shown) to provide power to the electrical components of the device 1.

A memory 7, preferably in the form of RAM, is connected to the central processing unit 4 and the central processing unit 4 is configured to read data from the memory 7 and write data to the memory 7. The memory 7 may be integrated into the device 1 or may take the form of a memory card which is releasably connected to the device 1.

An identifier reader 10 is connected to the central processing unit 4. The reader 10 is preferably an optical device which is operable to read identifiers in the form of coded patterns of dots provided on the surface of a data carrier and provide a signal indicative of the code defined by the dot pattern to the central processing unit 4. A coded dot pattern means the dot themselves and their relative positioning to one another.

Before the operation of the device 1 is discussed, types of data carrier on which the coded dot patterns may be formed will now be described.

Referring to FIG. 3 of the accompanying drawings, a data carrier for use with embodiments of the invention is in the form of a label 11. The label 11 is preferably of paper, but may instead be of a different material such as plastic. The label 11 is shown in FIG. 3 as a rectangular sheet, but the label 11 may alternatively be of any other shape.

The underside of the label 11, as seen in FIG. 3, is provided with an attachment means in the form of an adhesive layer (not shown) which allows the label 11 to be attached to an object. The adhesive layer may be formed of an adhesive which allows the label 11 to be adhered firmly to an object, or formed from an adhesive which allows the label 11 to be releasably attached to an object. In other arrangements, the label 11 is provided with an attachment means in the form of one of a hook layer and a loop layer of a hook and loop attachment arrangement to permit the label 11 to be releasably attached to the other hook or loop layer of the hook and loop attachment arrangement. In a further arrangement, the attachment means of the label 11 is defined by a magnetic layer which allows the label 11 to be releasably attached by magnetism to a ferrous object.

The upper side of the label 11, as seen in FIG. 3, is provided with an identifier. The identifier is formed by one coded dot pattern or a plurality of identical coded dot patterns 12 which are printed repeatedly on the upper surface of the label 11. The coded dot patterns 12 are spaced apart from one another to cover a region of the label 11. The spaced coded dot patterns 12 preferably cover the entire upper side of the label 11, but in some embodiments the coded dot patterns 12 may only cover a region forming part of the upper side of the label 11. Within each label, the coded dot patterns 12 are identical. This means that the entire region which is covered with the coded dot patterns 12 represents a single identifier, regardless of the location on the label 11.

The dots 12 are preferably formed by ink which is not visible to the naked eye, but which can be detected by the reader 10. The coded dot patterns 12 might, for instance, be formed from an ink which reflects light at a wavelength that is not visible to the human eye, such as a wavelength in the infrared or ultraviolet spectrum. The coded dot patterns 12 are preferably defined by a printed layer which is sufficiently thin such that the coded dot patterns 12 are not detectable by human touch. It is to be appreciated, however, that in some embodiments the coded dot patterns 12 may be visible to the naked eye and/or may be detected by human touch.

It is to be appreciated that, whilst FIG. 3 only shows one label 11, a plurality of difference labels 11 are preferably used with the device 1. The plurality of labels 11 may be identical in shape, but at least two of the labels 11 are provided with different identifiers. Each identifier is formed by a region of identical coded dot patterns with each coded dot pattern representing the identifier. Each label is provided with a unique identifier in the form of a unique set of coded dot patterns so that the labels 11 may be distinguished from one another by reading any one of the coded dot patterns within that label using the reader 10 of the device 1.

The label 11 is preferably blank and of a material such that the label 11 can be written or drawn on with ink. In other arrangements, however, the label 11 may not be blank, but may instead be coloured or be provided with pre-printed text and/or drawings on the side of the label 11 carrying the coded dots 12. It is to be appreciated that the coded dots 12 can be read by the reader 10 regardless of whether the label 11 is blank or carries ink or pre-printed text or drawings.

The operation of the system will now be described with reference to FIG. 4.

The device 1 remains in an inactive state until the activation switch 5 is pressed by a user. When the activation switch 5 is pressed, the central processing unit 4 is activated to sense the position of the mode switch 6.

If the record mode is selected using the mode switch 6, and the activation switch 5 is pressed and held down, the device 1 records a sound clip, such as words spoken by the user, by converting an analogue amplified signal from the microphone 2 into a digital sound clip and storing the sound clip in the memory 7. The sound recording is terminated when the user releases the activation switch 5.

The reader 10 is activated when sound recording commences. During or shortly after the recording of the sound clip, the user positions the device 1 so that the reader 10 is close to a data carrier provided with a coded dot pattern so that the reader 10 can read the coded dot pattern.

If the reader 10 is successful in reading a coded dot pattern then the reader 10 sends a signal indicative of the coded dot pattern to the central processing unit 4. The reader 10 is then deactivated. The central processing unit 4 processes the signal from the reader 10 and generates link data which is subsequently stored in the memory 7. The link data represents a link between the sound clip recorded in the memory 7 and the signal indicative of the coded pattern read by the reader 10. The link data is preferably data which is tagged onto the digital sound clip in the memory 7. However, in other arrangements the link data may be stored in another form in the memory 7, such as a lookup table. The purpose of the link data will become clear from the description below.

The central processing unit 4 preferably incorporates a timer which measures the length of time during which the reader 10 attempts to read a coded dot pattern during the record mode. The central processing unit 4 is configured to allow the reader 10 to continue attempting to read a coded dot pattern for a predetermined length of time.

If the reader 10 has not read a coded dot pattern after the predetermined length of time has elapsed then the timer times out. If the timer times out then the central processing unit 4 retrieves an error sound clip from the memory 7 and plays the error sound clip through the loudspeaker 9 to indicate to the user that the device 1 has not been able to link the recorded sound clip to a coded dot pattern. The error sound clip might, for instance, be in the form of a voice speaking an error message. It is to be appreciated, however, that in other arrangements the timer is omitted and the device 1 is not configured to play an error message. In some embodiments, the device 1 is provided with a warning light which is switched on when the device 1 is not able to correctly record a sound clip and link the sound clip with a coded dot pattern.

The operation steps in the record mode are complete once a sound clip has been recorded into the memory 7 and once link data has been saved into the memory 7. Once the record mode is complete, the user can switch the mode switch to the playback position so that the device 1 can operate in the playback mode.

If the activation switch 5 is pressed when the mode switch is positioned in the playback position then the central processing unit 4 activates the reader 10. Whilst the user presses the activation switch 5, the user positions the device 1 so that the reader 10 is adjacent a data carrier which is provided with a coded dot pattern. The reader 10 attempts to read the coded dot pattern whilst the activation switch remains pressed. If the reader 10 is successful in reading a coded dot pattern then the reader 10 sends a signal indicative of the coded dot pattern to the central processing unit 4. The central processing unit 4 processes the signal and searches the memory 7 for link data which corresponds with the particular coded dot pattern read by the reader 10. If the central processing unit 4 finds link data which corresponds to the coded dot pattern in the memory 7 then the central processing unit 4 retrieves the sound clip corresponding to the link data from the memory 7 and plays the sound clip to the user via the loudspeaker 9. The user can thus listen to the sound clip which the user recorded previously in association with the coded dot pattern.

If the central processing unit 4 does not identify link data corresponding to the coded dot pattern in the memory 7 then the central processing unit 4 terminates the playback operation mode until the activation switch is pressed again. If the activation switch 5 is pressed continually by the user then the device 1 remains in the playback mode and the reader 10 continues to attempt to read an identifier.

It is to be understood that the device 1 can be used to record multiple sound clips to build up a library of custom-made sound clips in the memory 7. The device 1 associates each sound clip with a different identifier by saving multiple sets of link data in the memory 7.

The device 1 is configured to be used with a plurality of data carriers which each carry an identifier which is unique from the identifiers on the other data carriers. Each identifier is independent from the other identifiers and is not related to the other identifier by location or otherwise. In one embodiment a plurality of labels 11 are each provided with a different identifier. The device 1 is used to record a sound clip associated with each label 11 and to save link data linking the identifier on that label 11 to the sound clip.

The user attaches each label 11 physically to a different object to effectively label each object with a sound clip which can be played back by the device 1. For instance, a person attaches a first label 11 to a first object. The user uses the device 1 to record a first sound clip which might be the user describing vocally the first object. The user is then able to use the device 1 to read the identifier on the first label 11 and associate the first sound clip with the first object.

The user attaches a second label 11 to a second object and use the device 1 to record a second sound clip which might be the user describing vocally the second object. The user can then use the device 1 to read the identifier on the second label 11 to associate the second sound clip with the second object.

Once the first and second sound clips have been recorded, the user uses the device 1 to identify which is the first object and which is the second object by activating the device 1 in the playback mode to play the sound clips associated with the labels on each object. A visually impaired person can thus effectively label everyday objects with audio notes so that the person can distinguish between the objects by hearing alone.

Since a user records the audio tags, the user can record audio descriptions in their own words and in a way which can be understood best by the user. This allows the system to be very flexible because the user is not limited to using third party audio descriptions which may be inaccurate or difficult to understand. That said, a person known to a user, such as a carrier, could also use the device 1 to record sound clips on behalf of a user and the user can use the device 1 to play back the sound clips.

It is to be appreciated that the labels 11 may be attached to any object, for example, commercial products, containers for medicine, walls or doors of buildings, electrical equipment. The labels 11 could, for instance, be attached at different positions on a document or in a book to effectively label parts of the document or book with different sound clips. The book might, for instance, be a diary and labels 11 could be attached to pages of the diary at positions representing a date or time so that the user can use the diary to organise their time by recording sound clips as entries in the diary.

Whilst the embodiment described above incorporates a physical mode selection switch 6 to switch the device 1 between the recording mode and the playback mode, in other embodiments, the mode of operation of the device 1 may be selected differently. For instance, in other embodiments, the device 1 may be configured to recognise voice commands spoken by a user to change the mode of operation of the device 1.

A device 1 in accordance with other embodiments of the invention is equipped with voice recognition and playback facilities which form an audio user interface (AUI). The audio user interface communicates with the user audibly by outputting speech via the loudspeaker 9 to tell the user the status of the device 1. The device 1 is also equipped with voice recognition so that the user can change the mode of operation and navigate through the audio user interface by speaking commands to the device 1.

Referring now to FIG. 5 of the accompanying drawings, the data carrier may be a document 13 which is designed to be used with the device 1. The document 13 has four regions A-D which may be blank or may be printed with text, drawings or photographs. The regions A-D each define regions of the document 13 which carry different identifiers. The identifier carried by each region A-D is uniform across that region A-D. The identifier carried by each region A-D is preferably defined by duplications of one coded dot pattern that is unique to that region which are printed repeatedly at spaced apart positions across the region. The identifier associated with one region A-D is independent from the identifiers provided on the other regions A-D and is not related to the other identifiers by location or otherwise. The arrangement is such that the device 1 will read only one identifier when the reader 10 is activated and positioned adjacent a region, regardless of where on the region the reader 10 is positioned.

A user can use the device 1 to record sound clips and associate each sound clip with an identifier carried on one of the regions A-D. A user can thus create an audio version of the document in their own words so that the device 1 can play the sound clip associated with the identifier of each region A-D selectively when the device 1 is used in the playback mode. It is to be appreciated that the document 13 may have a greater or fewer number of regions carrying identifiers than the four regions A-D shown in FIG. 5 and the document 13 may comprise several sheets.

In one arrangement, the document 13 is provided with symbols which can be detected by human touch, such as raised Braille characters. An identifier in the form of a coded dot pattern is printed so as to be superimposed on the symbols. A user can record a sound clip in the device 1 and link the sound clip to a symbol on the document 13 by using the device 1 to link the coded dot pattern associated with the symbol. This arrangement could, for instance, be used to teach a visually impaired person how to read Braille. A teacher can record a sound clip of themself saying the word represented by Braille characters and a pupil can practice reading the Braille characters and use the device 1 to listen to the meaning of the Braille characters as spoken by the teacher. The ability for a teacher to record their own sound clips allows the system to be flexible because the teacher can use their own voice to record the sound clips and offer any explanation, where necessary, which is tailored to a particular pupil.

An electronic aid comprising the device 1 and the document 13 may also have applications for assisting able-sighted people. For instance, the system could be used to teach reading skills. In this case, a teacher could record a sound clip of themself speaking a word and associate the sound clip with a identifier superimposed on a word printed on the document 13. A pupil can attempt to read the word and then see if they are correct by using the device 1 to hear the teacher saying the word. The document 13 could, initially, be a blank document, which is provided with a plurality of identifiers, each in the form of regions of invisible coded dot patterns. In this case, the teacher could write words or draw drawings on the document 13 at different positions and then record sound clips associated with the writing or drawing. The teacher can thus use the system as a flexible teaching tool which can be customised to teach a pupil speaking or reading skills as the teacher sees fit.

Referring now to FIG. 6 of the accompanying drawings, a further electronic aid incorporates the device 1 and a data carrier in the form of a diary 14. The diary 14 is laid out in a normal manner, with each page representing a date or time. Regions A-F of the diary 14 each represent a date or time and each region A-F is provided with different identifier which is printed in the diary 14. A user, such as a visually impaired person, can effectively make audio entries in the diary 14 by recording sound clips and linking each sound clip with an identifier in the diary 14 at a position indicative of a date or time. The system thus allows a visually impaired person to make use of the benefits of a diary to organise their time, relying on audio entries, rather than entries written in the diary. It is to be appreciated that the diary 14 may be provided with symbols, such as Braille, which may be detected by human touch to permit a visually impaired person to identify the different sections of the diary 14 by touch so that audio entries can be linked to regions of the diary 14 which are indicated with the touch-detectable symbols.

Referring now to FIG. 7 of the accompanying drawings, in another arrangement, there are two data carriers in the form of a product A and a product B. Products A and B are each provided with identifiers in the form of coded dot patterns printed directly on the products. The coded dot patterns might, for instance, be printed on the products A,B at the time when the products A,B were manufactured. Alternatively, instead of the coded dot patterns being printed on the products A,B, the coded dot patterns could be formed on the surfaces of products A,B when the products A,B are manufactured. The device 1 can be used to associate recorded sound clips with the products A,B. The user can then use the device 1 to listen to the sound clips to distinguish between the products A and B.

Whilst the arrangements described thusfar incorporate identifiers in the form of coded dot patterns, in other arrangements, an identifier can be defined by printed text and/or graphics which are visible to the human eye. In further arrangements, an identifier may be in the form of a symbol which is detectable by human touch, such as a Braille character. In yet further arrangements, an identifier may be in the form of magnetically encoded information. It is to be appreciated that in each arrangement, the identifier reader 10 is of a type capable of reading the form of identifier which is being used with the device 1.

In a further embodiment of the invention the device 1 is configured to store pre-recorded sound clips in the memory 7 and play back the pre-recorded sound clips in response to the reader 10 reading an identifier. The pre-recorded sound clips are recordings which are made using equipment other than the device 1. The pre-recorded sound clips are loaded into the memory 7 by connecting the device 1 to a computer, for example via a universal serial bus connection, a wireless connection or another suitable connection means. The pre-recorded sound clips could take the form of an audio version of a book, for instance a story, play, poetry collection, guidebook, textbook, instruction manual, etc.

In a yet further embodiment the device 1 incorporates a card reader to receive a memory card to provide a memory in addition to the internal memory 7 of the device 1. Memory cards for use with such embodiments are configured store pre-recorded sound clips which can be accessed and played back by the device 1 when the memory card is inserted into the card reader of the device 1.

The embodiments discussed above which are configured to store and play back pre-recorded sound clips are configured to be used with a book or document which is provided with a plurality of different identifiers at different positions. The device 1 plays back a particular sound clip, or a particular part of a sound clip, in response to the reader 10 reading a corresponding identifier on the book or document. For example, the device 1 can be used with a book which is blank except for a plurality of identifiers on regions of each page. The book may have a ribbon or another marking means to allow a user to mark their position in the book. A user can trace the reader 10 across the pages of the book in the same way that a person might use their finger to trace lines of words in a conventional book so that the reader 10 reads the identifiers successively.

The device 1 plays back a sound clip as the reader 10 reads the identifiers successively. If the user stops moving the device 1 and the reader 10 stops reading identifiers successively then playback of the sound clip is stopped. The user can resume playback of the sound clip from where they left off by continuing to move the device 1 across the page. It is to be appreciated that the identifiers in the book relate effectively to a position in the sound clip and that a user can choose to play back a particular part of a sound clip by tracing the reader 10 over identifiers at a particular position in the book. For example, a user can choose to listen to the start of a sound clip by tracing the reader 10 across identifiers at the start of the first page of the book, or to listen to the middle of a sound clip by tracing the reader 10 across identifiers on pages at the middle of the book. A user can thus play back different sections of the sound clip by pointing the reader 10 to different physical positions on pages of the book.

It is easy for a person to stop and resume listening to a sound clip by marking the page which they reached with the reader 10 physically, for instance using a bookmark or ribbon, and then subsequently finding the page and pointing the reader 10 to the position on the page where they left off. It is to be appreciated that this arrangement is beneficial for any person wishing to listen to an audio version of a book, not just blind or partially sighted persons. This arrangement is an improvement over conventional audio versions of books which are on compact discs because it offers an easier way to resume playback from a particular point, or skip to different points in a sound clip. Furthermore, the device 1 acts as the player for the audio version of the book, removing the need for a separate audio player such as a compact disc player.

The same book which is provided with identifiers may be used with the device 1 to play back a plurality of different sound clips. A user could thus store several different audio books in the memory of the device 1 and use the same identifier book to play back each audio book. This is advantageous because the user only has to carry one book and one device 1 to be able to listen to several different audio books which, if in printed form, would be bulky and inconvenient to carry.

It is to be appreciated that, whilst the embodiments described above are for use with a blank book that is provided with identifiers, in other arrangements the book may be not be blank and may instead be printed with text or drawings or carry tactile markers such as Braille characters.

It is to be appreciated that embodiments of the invention can be used by an able-sighted person to record sound clips as discussed above. Embodiments of the invention are as useful to an able-sighted person as they are to a blind or visually impaired person.

When used in this specification and claims, the terms “comprises” and “comprising” and variations thereof mean that the specified features, steps or integers are included. The terms are not to be interpreted to exclude the presence of other features, steps or components.

1. An electronic aid comprising a device which incorporates: a microphone for receiving an audio signal; an identifier reader for reading an identifier; a processing arrangement for receiving the audio signal from the microphone and creating a storable sound clip therefrom and for generating link data which defines a link between the sound clip and an identifier; a memory for storing the sound clip and link data corresponding to the sound clip; and an audio output; the device being configured to output the sound clip stored in the memory via the audio output in response to the identifier reader reading an identifier which is linked to the sound clip by corresponding link data stored in the memory.

2. An aid for use by a blind or visually impaired person, the aid comprising: a plurality of labels which are each provided with an attachment means to allow the label to be attached to an object, wherein at least two of the labels are provided with a respective different identifier; and a device which incorporates: a microphone for receiving an audio signal; an identifier reader for reading an identifier provided on one of the labels; a processing arrangement for receiving an audio signal from the microphone and creating a storable sound clip therefrom and for generating link data which defines a link between the sound clip and an identifier read by the identifier reader; a memory for storing the sound clip and link data corresponding to the sound clip; and
an audio output; the device being configured to output the sound clip stored in the memory via the audio output in response to the identifier reader reading the identifier which is linked to the sound clip by corresponding link data stored in the memory.

3. An aid according to claim 1, wherein the aid further comprises at least one data carrier which is provided with at least one identifier.

4. An aid according to claim 3, wherein the at least one data carrier is a label which is provided with an attachment means to allow the label to be attached to an object.

5. An aid according to claim 4, wherein the attachment means is an adhesive layer.

6. An aid according to claim 4, wherein the attachment means is one of a hook layer and a loop layer of a hook and loop attachment arrangement.

7. An aid according to claim 3, wherein the aid comprises a plurality of data carriers and wherein at least two of the data carriers are each provided with a different identifier.

8. An aid according to claim 3, wherein the at least one data carrier is a document which is provided with a plurality of different identifiers at different positions on the document.

9. An aid according to claim 3, wherein the at least one data carrier is a book which is provided with a plurality of different identifiers on different parts of the book.

10. An aid according to claim 9, wherein each identifier is unique to its position in the book.

11. An aid according to claim 10, wherein the device is configured to store in the memory a link between each of the identifiers and a part of a sound clip and to output a part of the sound clip in response to the identifier reader reading the identifier linked to the part of the sound clip.

12. An aid according to claim 10, wherein the device is configured to output parts of a sound clip successively while the identifier reader reads a plurality of the identifiers successively.

13. An aid according to claim 12, wherein the device is configured to stop outputting parts of the sound clip if the identifier reader stops reading the identifiers successively.

14. An aid according to claim 9, wherein the book is a diary and each identifier is provided on a respective part of the diary which is indicative of a date or a time.

15. An aid according to claim 3, wherein the at least one data carrier is provided with printed text and/or graphics in addition to the or each identifier.

16. An aid according to claim 3, wherein the at least one data carrier is provided with symbols which can be detected by human touch.

17. An aid according to claim 16, wherein the symbols are Braille characters.

18. An aid according to claim 1, wherein the or each identifier is a coded dot pattern.

19. An aid according to claim 1, wherein the or each identifier is a plurality of identical coded dot patterns which are spaced apart from one another.

20. An aid according to claim 18, wherein the or each coded dot pattern is printed.

21. An aid according to claim 18, wherein the or each coded dot pattern is not visible to the naked eye.

22. An aid according to any one of claims 18, wherein the or each coded dot pattern is not detectable by human touch.

23. An aid according to claim 18, wherein the coded dot pattern is detectable by human touch.

24. An aid according to claim 23, wherein the coded dot pattern is defined by at least one Braille character.

25. An aid according to claim 1, wherein the or each identifier is one of text and drawings which are visible to the naked eye.

26. An aid according to claim 1, wherein the identifier is magnetically encoded information.

27. An aid according to claim 1, wherein the device is configured to store a plurality of sound clips and a corresponding plurality of link data.

28. An aid according to claim 1, wherein the device is substantially pen-shaped.

29. An aid according to claim 1, wherein the audio output incorporates a loudspeaker.

30. An aid according to claim 1, wherein the audio output incorporates an earphone/headphone connection.

31. A method of storing information comprising: receiving an audio signal with a microphone, processing the audio signal with a processing arrangement to create a storable sound clip, reading an identifier provided on a data carrier using an identifier reader, generating link data which defines a link between the sound clip and the identifier, storing the sound clip and the link data corresponding to the sound clip in a memory.

32. A method of labeling an object comprising: attaching a label to an object, the label being provided with an identifier, reading the identifier using an identifier reader provided on a device, and recording a sound clip using the device, the device storing the sound clip and generating and storing link data which defines a link between the sound clip and the identifier so that the sound clip can be played back via an audio output in the device when the identifier reader reads the identifier when the device is subsequently activated in a playback mode.

33-34. (canceled)
